(19) United States
(12) Patent Application Publication
(10) Pub. No.: US 2005/0113925 A1
(43) Pub. Date: May 26, 2005

(54) Lumbar disc prostheses

(76) Inventor: Olivier Carli, Geneva (CH)

Correspondence Address: CARL M. NAPOLITANO, PH.D.;ALLEN, DYER, DOPPELT, MILBRATH & GILCHRIST, P.A., 255 SOUTH ORANGE AVE., SUITE 1401, P.O. BOX 3791, ORLANDO, FL, 32802-3791, US

(21) Appl. No.: 10/962,999

(22) Filed: Oct. 12, 2004

(30) Foreign Application Priority Data
Oct. 17, 2003 (FR) FR03/12144

(51) Int. Cl. A61F 2/44

(52) U.S. Cl. 623/17.13; 623/17.14

(57) ABSTRACT

A disk prosthesis for vertebrae includes two plates for attaching to adjacent vertebrae. A ball-and-socket joint is interposed between the two plates mounted atop one another and includes a spherical cap fitted onto an insert made of a ceramic material and mounted on one plate. A spherical cup operable with the spherical cap is fitted onto a second insert made of a ceramic material and mounted on the second plate. Each insert is mounted on a plate by means of a sliding link and a localized link to stop translation. The ball-and-socket joint is partly surrounded by an elastic membrane attached to the two plates for a linking one another.

### CROSS-REFERENCE TO RELATED APPLICATIONS

This application claims the benefit of French Application No. 03/12 144, filed Oct. 17, 2003, the disclosure of which is hereby incorporated herein in its entirety by reference.

### FIELD OF THE INVENTION

The invention covers a disk prosthesis for vertebrae, especially lumbar vertebrae, which is designed to replace the fibro-cartilaginous disks that connect the lumbar vertebrae of the spinal column.

### BACKGROUND OF THE INVENTION

It is known that an intervertebral disk can undergo alterations such as crushing, deformation, displacement or wear and, more generally, degeneration linked to mechanical stresses that are imposed on it and that lead to the anatomical and functional destruction of the disk and of the vertebral segment. This disk alteration modifies the mechanical performance of the disk and leads to a reduction in the height of the intersomatic space, which in turn entails a disturbance of the entire joint function. The result is instability that leads specifically to an arthrosic reaction, the source of pain and of osteophytic processes.

It has therefore been proposed to replace the defective disk with an artificial disk, for which different types of embodiment have been considered. For example, we are familiar from patent FR 2 594 882, with a disk prosthesis that involves a first and second plate for attachment to the neighboring vertebrae, made of a metallic material. A ball-and-socket joint is introduced between these two plates, which joint involves a first insert mounted on one of the plates consisting of a spherical cap that cooperates with the spherical cupule of a second insert mounted on the other plate. The inserts are in the form of disk slugs made for example of a bio-compatible ceramic material with improved tribologic characteristics, specifically as regards its resistance to wear. This type of prosthesis also has a shock absorbing ring around the ball-and-socket joint between the two plates.

While this type of lumbar prosthesis makes it possible to restore suitable height to the intersomatic space, the ball-and-socket joint of this prosthesis produces substantial friction on a small surface, thus making it sensitive to wear because of the important and localized stresses that the inserts undergo. In the same sense, the slugs are engaged in the attachment plates, during the operation of relaxing the distraction imposed on the vertebral bodies that receive the plates. It follows that a correct assembly is a relatively delicate process so that the ball-and-joint works under abnormal conditions.

### SUMMARY OF THE INVENTION

The present invention remedies drawbacks of earlier techniques by providing a prosthesis, a lumbar prosthesis by way of example, generally insensitive to wear and breakage phenomena, while being suitable for permitting physiological mobility between the two lumbar vertebrae so equipped. One prosthesis according to the invention may include a first and a second plate that are attached to neighboring vertebrae. A ball joint carried between the two plates mounted one atop the other may include a spherical cap arranged on a first insert made of a ceramic material and mounted on the first place and a spherical cupule working with the spherical cap and arranged on a second insert made of a ceramic material and mounted on the second plate. Each insert is mounted on a plate with the help of a sliding link and a localized link that stops translation. The ball joint is at least partially surrounded by an elastic membrane attached to the two plates in such a way that they are connected to one another.

In one embodiment, the sliding link may be a dovetailing type link between a plate and an insert. In an alternate embodiment, each localized link may comprise a stop lug traversing a bore hole made in a plate and opening onto a blind hole made in an insert.

The disk prosthesis may include a shock absorbing element interposed between each insert and a plate. Further, the spherical cupule and spherical cap may have substantially identical dimensions and be slightly less than the dimensions of the plates.

### BRIEF DESCRIPTION OF THE DRAWINGS

Various other characteristics will become clear from the description given below in reference to the attached drawings which show, as non-limiting examples, some exemplified embodiments of the invention, in which:

FIG. 1 is a perspective view showing an exemplified embodiment of a prosthesis according to the invention;

FIG. 2 is a partially blown up view showing a prosthesis according to the invention;

FIG. 3 is a cutaway front view substantially in the sagittal plane; and

FIG. 4 is a cutaway front view substantially in a transversal plane of a prosthesis according to the invention.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout, and prime notation is used to indicate similar elements in alternate embodiments.

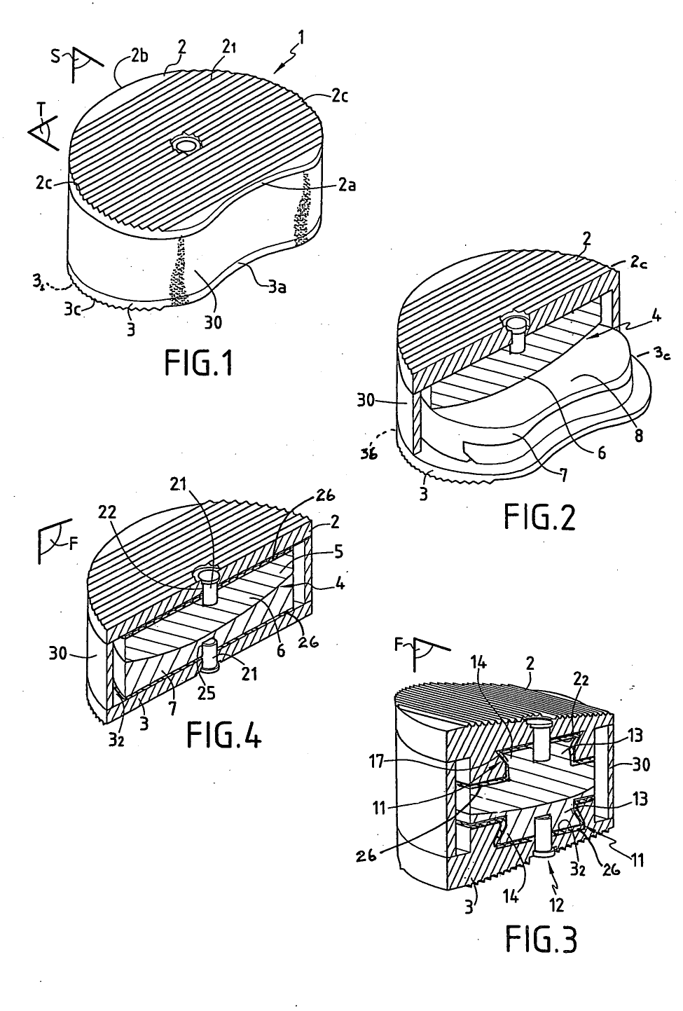

Referring initially to FIGS. 1 to 4, the invention covers a prosthesis, specifically a lumbar prosthesis 1, intended to be implanted in place of a disk between two adjacent vertebrae. The disk prosthesis 1, according to the invention, includes a first plate 2, called the upper plate in the example illustrated, and a second plate 3, known as the lower plate. Plates 2, 3 are intended to be attached to adjacent vertebrae and each one has an external face, respectively 2₁, 3₁ of substantially similar dimensions and adapted so as to approximately hug the contour of the associated articulating surfaces. Each plate 2, 3, also has an internal face, respectively 2₂ and 3₂, extending such that they are opposite one another. Each plate 2, 3 has a back edge, respectively 2a, 3a, extending opposite a front edge 2b, 3b respectively. The front edge 2b, 3b of each plate 2, 3 is connected to the respective back edge 2a, 3a by two lateral edges 2c, 3c, opposite one another. Preferably, in a transversal plane T perpendicular to the sagittal plane S with respect to the anatomical planes, the back edge 2a, 3a of each plate has a concave profile, whereas the front edge 2b, 3b has a convex profile. In this same transversal plane T, the lateral edges 2c, 3c also have a convex profile.

The disk prosthesis 1, as herein described by way of example, also includes a ball-and-socket joint 4, placed between the two plates 2 and 3, mounted atop one another. The ball-and-socket joint 4 comprises a first insert 5 having a spherical cap 6 and a second insert 7 that has a spherical cupule 8 that cooperates with the spherical cap 6. To be sure, the spherical cap 6 has a convex shape that is complementary to the concave shape of the spherical cupule 8. According to one advantageous embodiment characteristic, the spherical cap 6 and the spherical cupule 8 have substantially identical dimensions that are slightly less than the dimensions of the plates. It must be understood that the surfaces in contact between the two inserts have optimum dimensions that make it possible to distribute the constraints imposed. In one embodiment, the inserts 5, 7 are made of a ceramic material.

For the embodiment herein described, by way of example, each insert 5, 7 is mounted on a plate 2, 3 using a sliding link 11 and a localized 12 link to stop translation. In an exemplified embodiment, the sliding link 11 is a dovetailing type connection link made between a plate and an insert. As more precisely shown in FIGS. 2 and 3, to make this dovetailing type connection 11, each insert 5, 7 includes a projection 13 protruding from its spherical cap 6 and cupule 8 that terminates in a dovetailing part 14. As herein described, the dovetailing profile extends in a direction parallel to the frontal plane F. Each plate 2, 3 also includes on its internal face 22 and 23 a dovetailing profile 17, complementing the dovetailing profile of the inserts 5, 7. As more precisely appears from FIGS. 3 and 4, each insert 5, 7 is blocked in translation in the frontal direction in the example illustrated, through the localized link 12, consisting of a stop lug 21, that traverses a bore hole 22, made in each plate 2, 3 and opening into a blind hole 25 made in each insert. In this way, an effective and safe blocking of the insert is obtained while permitting an insert to be made whose dimensions are sufficient to cope with the efforts due to the axial stresses imposed on the prosthesis.

For one embodiment, as illustrated with reference again to FIG. 4, by way of example, a shock absorbing element 26 is interposed between each insert 5, 7 and cooperating plates 2, 3 for absorbing axial stresses imposed on the prosthesis 1.

In yet another embodiment, the prosthesis 1 may also include an elastic membrane 30 that, at least partially, covers the ball-and-socket joint 4, and is attached to the two plates 2, 3 so that the latter are interconnected. This elastic membrane 30 made, for example, of a polymer material, is attached by an appropriate means to the plates 2, 3 using an adhesive or a bonding, by way of example. This elastic membrane 30 makes it possible to maintain between them the two attachment plates 2, 3. Preferably, this elastic membrane 30 continues all around the circumference of the prosthesis, which, therefore, has the form of a completely closed element.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A disk prosthesis comprising:
a first plate and an opposing second plate each having a surface for attaching to adjacent vertebrae;
a ball-and-socket joint operable between the two plates, the ball-and socket joint including a spherical cap carried on a first insert made of a ceramic material and mounted on the first plate, and a spherical cupule operable with the spherical cap and carried on a second insert made of a ceramic material and mounted on the second plate;
first and second sliding links and localized links operable with each of the first and seconds inserts, and the first and second plates, respectively, for limiting a translation thereof; and
an elastic membrane linking the first and second plates and extending at least partially around the ball-and-socket joint.

2. A disk prosthesis according to claim 1, wherein the sliding link comprises a dovetail link made between at least one of the first plate and the first insert, and the second plate and the second insert.

3. A disk prosthesis according to claim 1, wherein at least one of the first and second localized links comprises a stop lug traversing a bore hole made in the plate and an opening into a blind hole made in the insert.

4. A disk prosthesis according to claim 3, further comprising a shock absorbing element interposed between at least one of the first and second inserts and the first and second plates, respectively.

5. A disk prosthesis according to claim 4, wherein the spherical cupule and the spherical cap have substantially identical dimensions, which dimensions are slightly less than the dimensions of the first and second plates.

6. A disk prosthesis according to claim 1, further comprising a shock absorbing element interposed between at least one of the first and second inserts and the first and second plates, respectively.

7. A disk prosthesis according to claim 1, wherein the spherical cupule and the spherical cap have substantially identical dimensions, which dimensions are slightly less than the dimensions of the first and second plates.

8. A disk prosthesis comprising:
a first plate and an opposing second plate each having a surface for attaching to adjacent vertebrae;
a ball-and-socket joint operable between the two plates, the ball-and socket joint including a spherical cap carried on a first insert made of a ceramic material and mounted on the first plate, and a spherical cupule operable with the spherical cap and carried on a second insert made of a ceramic material and mounted on the second plate; and
an elastic membrane linking the first and second plates and extending at least partially around the ball-and-socket joint.

9. A disk prosthesis according to claim 8, further comprising at least one of first and second sliding links and localized links operable with each of the first and seconds inserts, and the first and second plates, respectively, for limiting a translation thereof.

10. A disk prosthesis according to claim 9, wherein at least one of the sliding links comprises a dovetail link made between at least one of the first plate and the first insert, and the second plate and the second insert.

11. A disk prosthesis according to claim 9, wherein at least one of the first and second localized links comprises a stop lug traversing a bore hole made in the plate and an opening into a blind hole made in the insert.

12. A disk prosthesis according to claim 8, further comprising a shock absorbing element interposed between at least one of the first and second inserts and the first and second plates.

13. A disk prosthesis according to claim 8, wherein the spherical cupule and the spherical cap have substantially identical dimensions, which dimensions are slightly less than the dimensions of the first and second plates.

14. A disk prosthesis comprising:
a first plate and an opposing second plate each having a surface for attaching to adjacent vertebrae;
a ball-and-socket joint operable between the two plates, the ball-and socket joint including a spherical cap carried on a first insert made of a ceramic material and mounted on the first plate, and a spherical cupule operable with the spherical cap and carried on a second insert made of a ceramic material and mounted on the second plate; and
first and second sliding links and localized links operable with each of the first and seconds inserts, and the first and second plates, respectively, for limiting a translation thereof.

15. A disk prosthesis according to claim 14, further comprising an elastic membrane linking the first and second plates and extending at least partially around the ball-and-socket joint.

16. A disk prosthesis according to claim 14, wherein the sliding link comprises a dovetail link made between at least one of the first plate and the first insert, and the second plate and the second insert.

17. A disk prosthesis according to claim 14, wherein at least one of the first and second localized links comprises a stop lug traversing a bore hole made in the plate and an opening into a blind hole made in the insert.

18. A disk prosthesis according to claim 14, further comprising a shock absorbing element interposed between at least one of the first and second inserts and the first and second plates, respectively.

19. A disk prosthesis according to claim 14, wherein the spherical cupule and the spherical cap have substantially identical dimensions, which dimensions are slightly less than the dimensions of the first and second plates.
